(19) United States
(12) Patent Application Publication
(10) Pub. No.: US 2021/0330922 A1
(43) Pub. Date: Oct. 28, 2021

(54) NEGATIVE PRESSURE ORONASAL APPARATUS

(71) Applicant: Government of the United States as represented by the Secretary of the Air Force, Wright-Patterson AFB, OH (US)

(72) Inventors: Andrew G. Boston, Silver Spring, MD (US); Mostafa M. Ahmed, Belton, TX (US); Thomas J. Wilson, Boerne, TX (US); Erik K. Weitzel, San Antonio, TX (US)

(73) Assignee: Government of the United States as represented by the Secretary of the Air Force, Wright-Patterson AFB, OH (US)

(21) Appl. No.: 16/985,829

(22) Filed: Aug. 5, 2020

Related U.S. Application Data
(60) Provisional application No. 63/013,991, filed on Apr. 22, 2020.

(51) Int. Cl. A61M 16/06; A61M 16/04; A61M 16/20; A61M 16/00

(52) U.S. Cl. CPC A61M 16/0622; A61M 16/0468; A61M 16/047; A61M 2209/088; A61M 16/208; A61M 16/205; A61M 16/009; A61M 16/0683

(57) ABSTRACT

A negative pressure surgical apparatus. The apparatus includes a mask configured to cover a portion of the face of a patient and including a first port and an instrument port. The first port is configured to draw negative pressure from between the mask and the face of the patient. The instrument port is configured to receive a medical instrument therethrough and form a seal therewith. The instrument port is positioned on the mask so as to permit surgery at a surgical site.

Pursuant to 37 C.F.R. § 1.78(a)(4), this application claims the benefit of and priority to prior filed co-pending Provisional Application Ser. No. 63/013,991, filed Apr. 22, 2020, which is expressly incorporated herein by reference in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to personal protective equipment for health care workers and, more particularly, to personal protective equipment worn by a patient for protecting the heath care worker.

### BACKGROUND OF THE INVENTION

Viruses, such as the Coronavirus (COVID-19), and other airborne pathogens may be highly concentrated in the nose of an infected patient. At times, a patient may be infected with, or recently exposed to, the virus but not yet exhibit symptoms of invention. During this time, it is possible that the patient may inadvertently expose nearby persons to the virus.

While societal practices during pandemic periods require social distancing or wearing of a facial covering to minimize potential for exposure, such practices are not possible or feasible during medical emergencies or medical procedures. For example, if a patient requires oral, nasal, sinus, or other similar surgery, it is possible the patient, being an infected person, could expose surgeons or other medical personnel assisting in the surgery to the virus. While pre-operative examinations and physicals may include testing for infectious diseases, false negatives or very recent exposures may provide a false sense of security.

Beyond the operation room, first responders are called to treat patients of unknown exposure status. Often there is not time to triage the patient, perform a basic physical, or test for infectious diseases. Thus, the potential for first responders to be exposed to airborne pathogens remains very high.

There exists a need for devices and methods that may be utilized to reduce or eliminate the potential for exposing healthcare providers to airborne pathogens.

### SUMMARY OF THE INVENTION

The present invention overcomes the foregoing problems and other shortcomings, drawbacks, and challenges of limiting the exposure of health care workers while treating a patient of unknown exposure status. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention a negative pressure surgical apparatus that includes a mask configured to cover a portion of the face of a patient and that includes a first port and an instrument port. Both ports extend through the mask. The first port is configured to draw negative pressure from between the mask and the face of the patient. The instrument port is configured to receive a medical instrument therethrough and form a seal therewith. The instrument port is positioned on the mask so as to permit surgery at a surgical site.

Another embodiment of the present invention includes a negative pressure surgical apparatus that includes a mask configured to cover a portion of the face of a patient. First, second, and third ports extend through the mask and are configured to draw a negative pressure, provide gas to a patient, and exhaust exhaled breath, respectively. An instrument port extends through the mask and is configured to receive a medical instrument therethrough and form a seal therewith, the instrument port positioned on the mask so as to permit surgery at a surgical site.

Still another embodiment of the present invention is directed to a method of performing oronasal surgery by positioning a negative pressure surgical apparatus on the face of a patient. The negative pressure surgical apparatus includes a mask configured to cover a portion of the face of a patient. A first port extends through the mask. An instrument port also extends through the mask and is configured to receive a medical instrument therethrough. The instrument port is also configured to form a seal with the medical instrument. The instrument port is positioned on the mask so as to permit surgery at a surgical site. A seal is formed between the face of the patient and the mask by drawing negative pressure through the first port. A medical instrument is inserted through the instrument port to the surgical site.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a front, elevation view of a negative pressure oronasal apparatus according to an embodiment of the present invention.

FIG. 2 is a partially exploded, side elevation view of the negative pressure oronasal apparatus of FIG. 1.

FIG. 3 is a bottom view of the negative pressure oronasal apparatus of FIG. 1.

FIG. 4 is a perspective view of the negative pressure oronasal apparatus of FIG. 1 worn by a patient according to an embodiment of the present invention.

FIG. 5 is a front, elevation view of a negative pressure oronasal apparatus according to another embodiment of the present invention.

FIG. 6 is a cross-sectional view taken along the line 6-6 of FIG. 5.

FIG. 7 is an enlarged, perspective view of an intubation aperture of the apparatus of FIG. 5.

FIG. 8 is a partially exploded, side elevation view of the negative pressure oronasal apparatus of FIG. 5.

FIG. 9 is a bottom view of the negative pressure oronasal apparatus of FIG. 5.

FIG. 10 is a perspective view of the negative pressure oronasal apparatus of FIG. 5 worn by a patient according to an embodiment of the present invention.

FIG. 11 is a front, elevation view of a negative pressure oronasal apparatus according to another embodiment of the present invention.

FIG. 12 is a front, elevation view of a negative pressure oronasal apparatus according to still another embodiment of the present invention.

FIG. 13 is a perspective view of a negative pressure oronasal apparatus according to another embodiment of the present invention worn by a patient.

FIGS. 14 and 15 are front, elevation views of negative pressure oronasal apparatuses according to yet other embodiments of the present invention.

FIGS. 16 and 17 are perspective views of negative pressure oronasal apparatuses incorporated into negative pressure hoods according to embodiments of the present invention and worn by patients.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

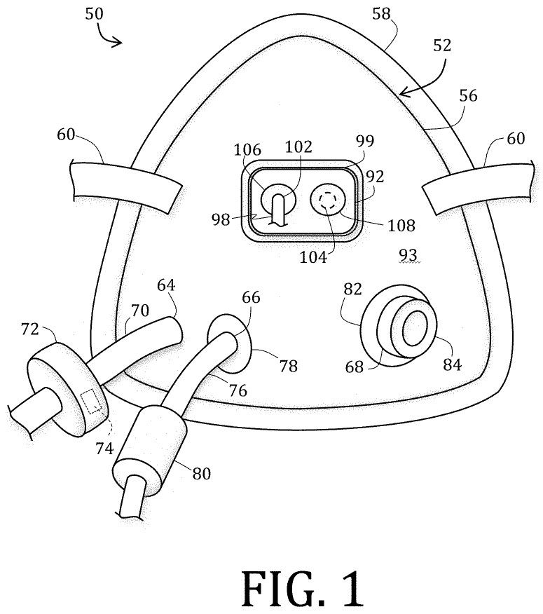

Referring now to figures, and in particular to FIGS. 1-4, an oronasal apparatus 50 according to an embodiment of the present invention is shown. The oronasal apparatus 120 includes a mask 52 that is configured to extend over the nose and the mouth of a patient 54. The mask 52 may be constructed from a moldable material, such as a polymer (one particular example being PVC, polyvinyl chloride), soft rubber, silicon or other biocompatible materials. The material may be partially pliable but should be sufficiently rigid to maintain shape and support negative pressure.

A perimeter 56 of the mask 52 may include a cuff 58 that is configured to form a seal between the mask 52 and the patient 54. The cuff 58 may be constructed from an open cell foam material (such as polyurethane), silicone, gel, foam, memory foam, and so forth. In some embodiments, the cuff 58 may be an adhesive tape or an inflatable bladder. The inflatable bladder embodiment may be constructed from polyurethane, latex, or other flexible and expandable plastic that may be filled with gas to expand and conform to the patient's face and form a seal therewith.

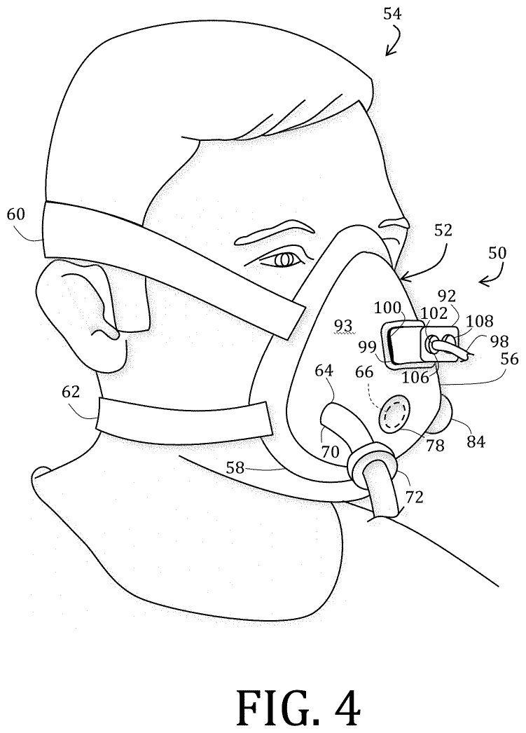

One or more straps (one strap 60 is shown in FIGS. 1-3; a second strap 62 is shown in FIG. 4) may be operably coupled to the mask 52 and are configured to secure the mask 52 to the patient 54. The straps 60, 62 may be elastic, fabric, or other suitable material. According to some embodiment, the straps 60, 62 may be stretchable so as to expand to fit certain patients; alternatively, in other embodiments, the straps 60, 62 may include a hook and loop material (such as VELCRO brand materials), a ratchet, a slidable adjuster, a tie down, or otherwise be tied to secure the mask 52.

The mask 52 may include a plurality of operable ports 64, 66, 68, each configured for a particular function during use of the mask 52. A first port 64 may be configured to be operably coupled to a medical vacuum (not shown) or other similar negative pressure device. The medical vacuum may be coupled to the first port 64 by way of a tube 70 and, optionally, a filter cartridge 72. The filter cartridge 72 may include a filter (not shown) configured to remove pathogens from air flowing through the tube 70 from the mask 52. Suitable filters may include but are not limited to N95, N99, N100, R95, P95, P99, and P100. In this way, after the mask 52 is positioned on the face of the patient 54 and the medical vacuum activated, air is pulled from between the patient 54 and the mask 52, through the tube 70 and filter cartridge 72 to form the seal and remove airborne pathogens from the pulled air. According to some embodiments, the filter cartridge 72 may further incorporate a sensor 74 operable to detect airborne pathogens and to alert the physician of such detection. Such sensors are commercially available.

A second port 66 of the mask 52 may be operably coupled to an oxygen or air supply (not shown) by way of a supply tube 76 and, optionally, a valve 78 (such as a check-valve, one-way valve, or flutter valve) so as to prevent reverse flow of air. In this way, oxygen may flow to the patient 54 and not in reverse. The second port 66 enables oxygen flow to the patient 54 while the negative pressure is applied to the mask 52. As with the tube 70 of the first port 64, the supply tube 76 may include a filter 80 operable to remove potentially harmful pathogens from the airflow to the patient 54.

A third port 68 of the mask 52 may be configured as an exhaust for removal of carbon dioxide not otherwise vented by way of the first port 64. This third port 68, like the second port 66, may include a valve 82 (such as a check-valve, one-way valve or flutter valve) configured to permit venting of carbon dioxide and to prevent inhalation of unfiltered air. The port 68 may further include a filter 84 configured to remove airborne pathogens from the vented air. In this way, the health care worker is protected from exposure. According to some embodiment, the third port 68 may include a positive end expiratory pressure valve in lieu of the one-way valve 82. The positive end expiratory pressure valve is operable to regulate the pressure in the lungs of the patient 54 to improve lung inflation. Specifically, the positive end expiratory pressure valve may be used to maintain pressure on the lower airways at the end of the breathing cycle, which prevents alveoli collapse during expiration.

Referring still to FIGS. 1-4, the mask 52 further includes a protrusion 90 and a corresponding cap 92. The protrusion 90 may be located on the mask 52 so as to facilitate nasal or oral surgery at a particular surgical site. Although not particularly shown in these embodiments, the protrusion 90 may taper toward a surface 93 of the mask 52 to facilitate installation and removal of the cap 92.

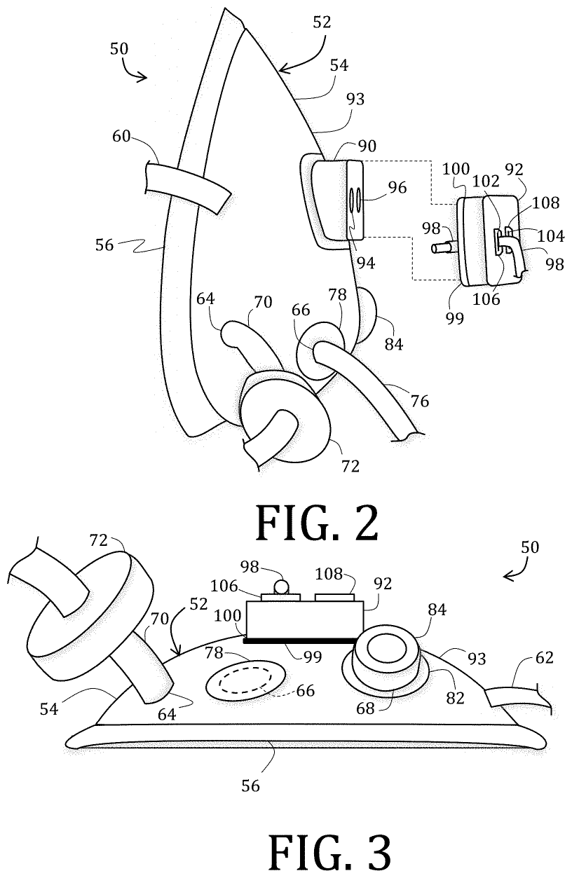

As specifically shown in FIG. 2, the protrusion 90 includes an instrument (two instrument ports 94, 96 are shown) that are shaped and sized to receive a medical instruments (one instrument 98 is shown). While the specific instrument depends on the particular surgery to be performed, some typical instruments may include a flexible laryngoscope, a flexible bronchoscope, a rigid endoscope, a nasal suction, or a flexible esophagoscope. The protrusion 90 may be formed with the mask 52, such as a unitary structure and thus of the same material as the mask 52. Alternatively, the protrusion 90 may be separately fabricated and then operably coupled to the mask 52, such as with an adhesive. If fabricated separately, then a plurality of protrusions may be fabricated, and an appropriate protrusion for the specific surgery coupled to the mask 25.

The cap 92 may be shaped and sized so as to be received by the protrusion 90 and constructed from rigid material. Suitable materials may include those listed above with respect to the mask 25. In some embodiments, the cap 92 may be secured to the protrusion 90 by friction fit, may include a rail (not shown) configured to snap fit with a channel (not shown) of the protrusion 90, or may include tabs (not shown) that are received by recesses (not shown) of the protrusion 90. Still other methods of securing the cap 92 to the protrusion 90 would be known to those of ordinary skill in the art having the benefit of the disclosure made herein. In the illustrated embodiment, although not required, the cap 92 may further incorporate a seal 99 on a distal end 100 thereof and that is configured to form a seal with the mask 52. Suitable sealant materials are known and may include, for example, silicon.

The cap 92 includes a port (two ports 102, 104 are shown) that coincide or align with instrument ports 94, 96 of the protrusion 90. In some embodiments, the ports 102, 104 of the cap 92 may be coaxial to the instrument ports 94, 96 of the protrusion 90. Each port 102, 104, such as those of the illustrated embodiment, includes a valve 106, 108 (such as a check-valve, one-way valve, or a flutter valve) configured to permit advancement and retraction of the medical instrument but not otherwise permitting exchange of air therethrough. Accordingly, the valves 106, 108 are configured to form a seal with the medical instrument, when present, and otherwise seal the ports 102, 104 when the medical instrument is not present.

With particular reference now to FIG. 4, use of the oronasal apparatus 50 according to the particular illustrated embodiment is shown. As noted above, the apparatus 50 may be positioned on a patient 54 having unknown exposure of airborne pathogens. The patient 54 may, for example, be in the process of being prepped for surgery, awaiting triage, admitted to in-patient care, undergoing intubation, undergoing esophagoscopy, or en route to medical care. The mask 52 is positioned over the nose and mouth of the patient 54 and the straps 60, 62 adjusted to secure fit.

A medical vacuum may then be operably coupled to the first port 64 while an air or oxygen supply may be coupled to the second port 66. Activating the medical vacuum draws a slight vacuum between the mask 52 and the face of the patient 54, which draws the cuff 58 to the face to form a seal. If exhalation exhaust beyond the third port 68 and filter 84, then tubing or other devices may be operably coupled through the third port 68.

If the patient 54 was prepped for surgery, then a procedure may be performed through the ports 94, 96 102, 104 of the protrusion 90 and the cap 92. If one embodiment of the cap 92 is insufficient, then another embodiment may be used. In some instances, the cap 92 may be removed and the procedure accomplished through the ports 94, 96 of the protrusion 90. Use of the apparatus 50 protects the healthcare workers during a procedure from potential exposure of undiagnosed airborne pathogens.

Otherwise, the apparatus 50 may be used on any patient that engages medical attention and that may have infections transmitted by airborne particulates. As such, no operation or procedure need be performed by way of the protrusion 90, with or without the cap 92.

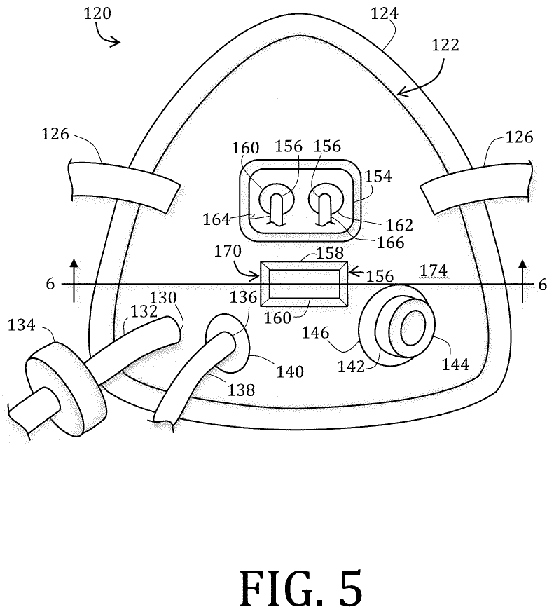

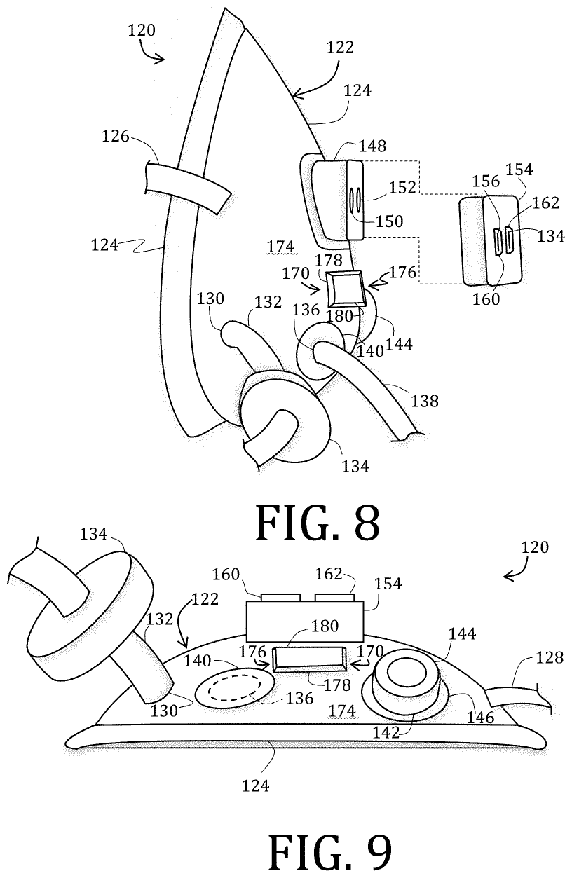

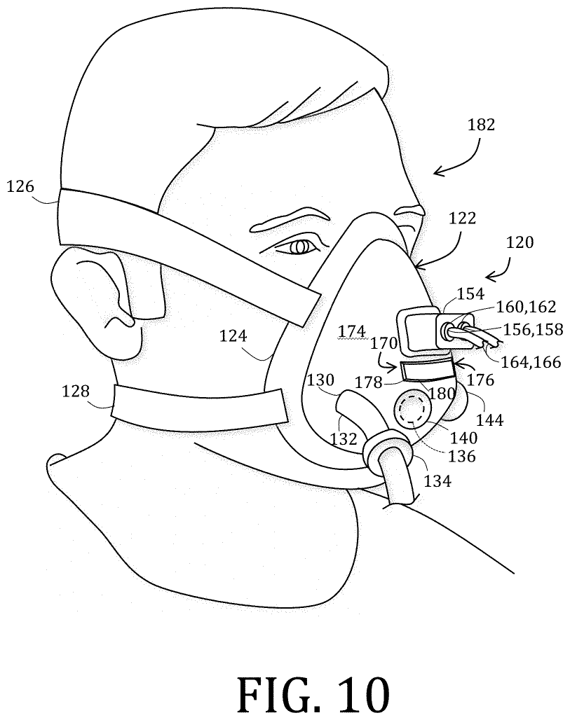

With reference now to FIGS. 5-10, an oronasal apparatus 120 according to another embodiment of the present invention is described. The particular apparatus 120 of this embodiment includes a mask 122, a cuff 124, and a strap (two straps 126, 128 are shown in FIG. 10), all of which may be constructed and fabricated in a manner similar to what was described with previous embodiments. Furthermore, and like the apparatus 50 of FIG. 1, the apparatus 120 according to this illustrative embodiment includes a first port 130 configured to be operably coupled to a medical vacuum (with tubing 132 and a filter 134), a second port 136 configured to be operably coupled to an air or oxygen supply (with a supply tube 138 and a valve 140), and a third port 142 configured to be operated as an exhaust (with a filter 144 and a valve 146). Additionally, as was described for the apparatus 50 of FIG. 1, the mask 122 further includes a protrusion 148 having a port (two ports 150, 152 are shown) and cap 154 (having two ports 156, 158 and associated filters 160, 162). The ports 150, 152, 156, 158 of the protrusion 148 and of the cap 154 are configured to receive a medical instrument for conducting a medical procedure therethrough (two instruments 164, 166 are shown in some illustrations).

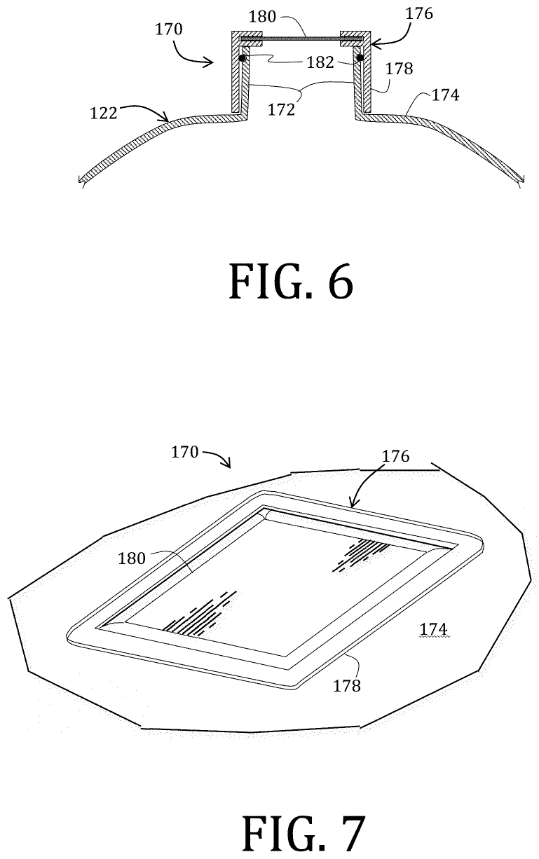

The particular embodiment of FIG. 5 (with reference to FIGS. 6 and 7), unlike the embodiment of FIG. 1, further includes an intubation aperture 170. The intubation aperture 170 may be fabricated as walls 172 extending away from a surface 174 of the mask 122 and defining an opening therebetween, as particularly illustrated in FIG. 6. According to an alternative embodiment, the opening may be cut into the mask 122 after fabrication and, therefore, does not incorporate walls 172.

The intubation aperture 170 may be configured to receive a window 176 comprising a frame 178 and a sheath 180 stretched within the frame 178. The window 176, as more particularly shown in FIGS. 6 and 7, may be secured by friction fit and sealed with an O-ring 182 (or a c-ring, for example). However, other arrangements may also be possible. For example, the window 176 may include a rail and channel snap fit, tabs received by recesses (not shown). The sheath 180 may be any material (such as a membrane, rubber, or silicone) that is self-healing or configured to form a seal while permitting passage of an endotracheal tube.

An advantage of the particular arrangement of FIGS. 5-7 is that the window 176 may be disposable while mask 122 is reusable. In this manner, the sheath 180 assists in maintaining the seal formed between the mask 122 and the patient 184 while still permitting advancing and retracting of medical instruments 164, 166.

The particular embodiment of FIGS. 5-10 may be useful when intubation is necessary during an operative procedure carried out through the ports 150, 152, 156, 158. Additionally, or alternatively, the embodiment may also facilitate the use of larger medical instruments that are not otherwise accommodated by the ports 150, 152, 156, 158.

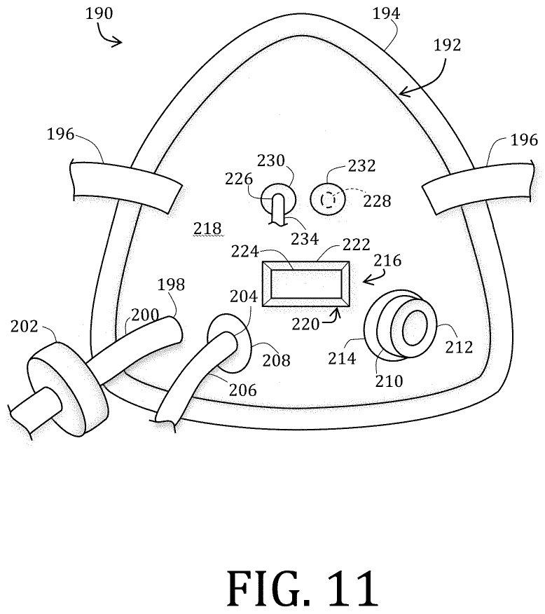

FIG. 11 illustrates yet another oronasal apparatus 190 according to another embodiment of the present invention. The particular apparatus 190 of this embodiment includes a mask 192, a cuff 194, and a strap 196, all of which may be constructed and fabricated in a manner similar to what was described with previous embodiments. Also like some other embodiments, the apparatus 190 includes a first port 198 configured to be operably coupled to a medical vacuum (with tubing 200 and a filter 202), a second port 204 configured to be operably coupled to an air or oxygen supply (with a supply tube 206 and a valve 208), and a third port 210 configured to be operated as an exhaust (with a filter 212 and a valve 214). The particular embodiment of FIG. 11 also includes an intubation aperture 216. The intubation aperture 216 may be fabricated as walls extending away from a surface 218 of the mask 192 and defining an opening therebetween (as particularly illustrated in FIG. 6), may be cut into the mask 192 after fabrication and, therefore, not incorporate walls, or fabricated in another way that that would understood by those of ordinary skill in the art having the benefit of this disclosure. The intubation aperture 216 may be configured to receive a window 220 comprising a frame 222 and a sheath 224 stretched within the frame 222. The window 220 may be secured by friction fit and sealed with an O-ring 182 (FIG. 6) (or a c-ring, for example), include a rail and channel snap fit, or tabs received by recesses (not shown). The sheath 224 may be any material (such as a membrane) that is self-healing while permitting passage of an endotracheal tube.

For the particular embodiment of FIG. 11, a surgical instrument port (two ports are shown 226, 228) may be fabricated directly into the surface 218 of the mask 192 in a manner similar to the ports 198, 204, 210 and without the protrusion 90 (FIG. 1). The ports 226, 228 may include valves 230, 232 in the manner described previously and configured to form a seal around a surgical instrument 234 when present or seal the port when not present.

An advantage of the particular arrangement of FIG. 11 is that the apparatus 190 may be used without a cap 92 (FIG. 1), i.e., consumable items.

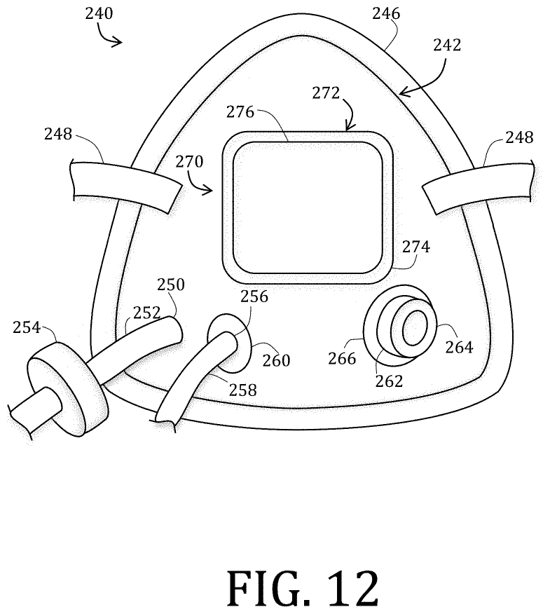

FIG. 12 illustrates still another oronasal apparatus 240 according to another embodiment of the present invention. The particular apparatus 240 of this embodiment includes a mask 242, a cuff 246, and a strap 248, all of which may be constructed and fabricated in a manner similar to what was described with previous embodiments. Also like some other embodiments, the apparatus 240 includes a first port 250 configured to be operably coupled to a medical vacuum (with tubing 252 and a filter 254), a second port 256 configured to be operably coupled to an air or oxygen supply (with a supply tube 258 and a valve 260), and a third port 262 configured to be operated as an exhaust (with a filter 264 and a valve 266).

The embodiment illustrated in FIG. 12 includes an enlarged intubation aperture 270, which may be constructed in one of the manners described previous (i.e., walls 172 extending from a surface 174 of the mask 122, an opening cut into the surface 174 of the mask 122, and so forth). As shown, the intubation aperture 216 is configured to receive a window 272 comprising a frame 274 and a sheath 276 stretched within the frame 274. The window 272 may be secured by friction fit and sealed with an O-ring 182 (FIG. 6) (or a c-ring, for example), include a rail and channel snap fit, or tabs received by recesses (not shown). The sheath 276 may be any material (such as a membrane) that is self-healing while permitting passage of an endotracheal tube.

Advantages of the illustrative embodiment of FIG. 12 is that the apparatus 240 may be used any size medical instrument and a plurality of instruments may be manipulated within the same port or opening.

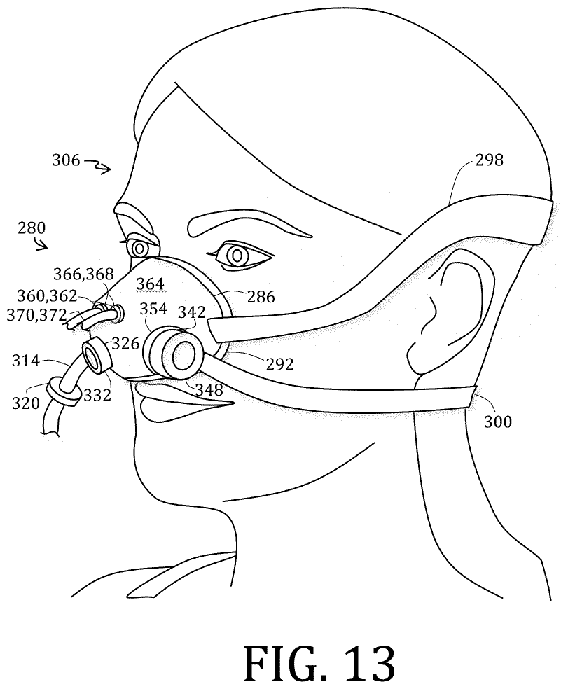

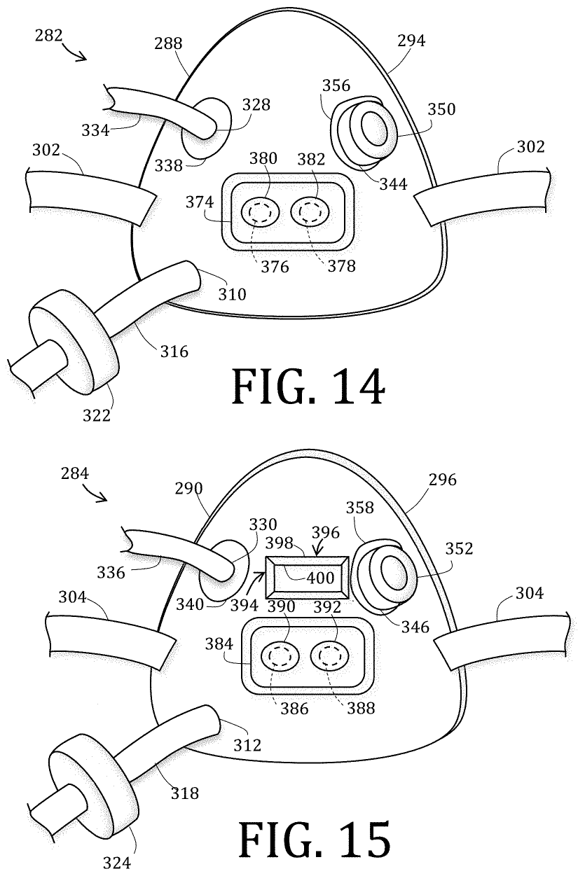

FIGS. 13-15 illustrate oronasal apparatuses 280, 282, 284 according to other embodiments of the present invention. The particular illustrated embodiments of these figures may be useful in limited nasal procedures or for a patient that does not tolerate other embodiments of the oronasal apparatus covering the nose and mouth. Each of these embodiments respectively includes a mask 286, 288, 290, a cuff 292, 294, 296, and a strap (two straps 298, 300 are shown in FIG. 13 while FIGS. 14 and 15 each include a single strap 302, 304). Unlike previously described embodiments, the apparatuses of these figures are shaped so as to cover the nose of a patient 306.

Each mask 286, 288, 290 further includes, similar to other embodiments, a first port (not shown in FIG. 13), 310, 312 configured to be operably coupled to a medical vacuum (with tubing 314, 316, 318 and a filter 320, 322, 324), a second port 326, 328, 330 configured to be operably coupled to an air or oxygen supply (with a filter 332 in FIG. 13 and supply tube 334, 336 and a valve 338, 340 in FIGS. 14 and 15), and a third port 342, 344, 346 configured to be operated as an exhaust (with a filter 348, 350, 352 and a valve 354, 356, 358).

The mask 286 of FIG. 13 further includes a surgical instrument port (two ports 360, 362 are shown) may be fabricated directly into a surface 364 of the mask 286 in a manner similar to the ports 226, 228 (FIG. 11) without the protrusion 90 (FIG. 1). The ports 360, 362 may include valves 366, 368 in the manner described previously and configured to form a seal around a surgical instrument (two instruments 370, 372 are shown) when present or seal the port 360, 362 when not present.

The mask 288 of FIG. 14 provides an alternate arrangement of the first, second, and third ports 310, 328, 344 and further incorporates a protrusion (not shown in FIG. 14) and a corresponding cap 374 having two instrument ports 376, 378 with valves 380, 382, which may be fabricated and used in accordance with other embodiments described herein.

In FIG. 15, the illustrated embodiment demonstrates the alternate arrangement of the first, second, and third ports 312, 330, 346, a protrusion (not shown in FIG. 15), and a cap 384 with instrument ports 386, 388 and valves 390, 392 similar to the apparatus 284 282 of FIG. 14. The particular apparatus 284 of FIG. 15 further incorporates an intubation aperture 394. The intubation aperture 394 may be fabricated as walls 172 (FIG. 6) extending away from a surface 174 (FIG. 6) of the mask 122 (FIG. 6) and defining an opening therebetween, may be cut into the mask 290 after fabrication and, therefore, not incorporate walls, or fabricated in another way that that would understood by those of ordinary skill in the art having the benefit of this disclosure. The intubation aperture 394 may be configured to receive a window 396 comprising a frame 398 and a sheath 400 stretched within the frame 398. The window 396 may be secured by friction fit and sealed with an O-ring 182 (FIG. 6) (or a c-ring, for example), include a rail and channel snap fit, or tabs received by recesses (not shown). The sheath 400 may be any material (such as a membrane) that is self-healing while permitting passage of an endotracheal tube.

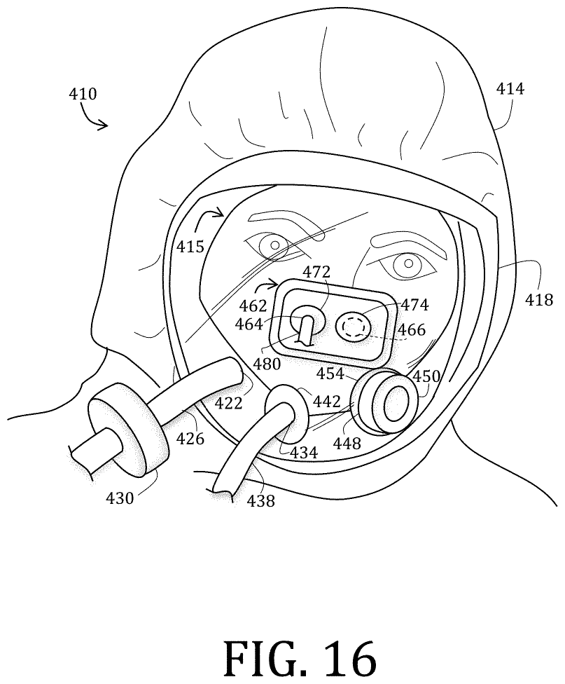

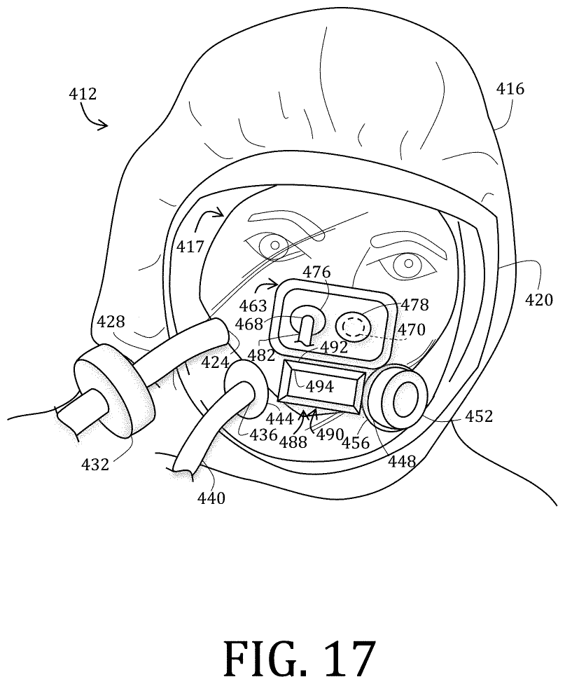

FIGS. 16 and 17 illustrate oronasal apparatuses 410, 412 according to yet other embodiments of the present invention. As shown in these embodiments, each oronasal apparatus 410, 412 is incorporated into a negative pressure hood 414, 416 for use by a patient 415, 417. The negative pressure hood 414, 416 may be constructed from a non-porous material that is configured to resist transport of airborne pathogens therethrough. According to some embodiments, the hood 414, 416 may further incorporate a cape (not shown) to cover the patient torso or may be a full gown or bodysuit. According to still other embodiments, the negative pressure hood 414, 416 may be incorporated into a biohazard suit. The negative pressure hood 414, 416 includes a facemask 418, 420 that may be constructed from a shatterproof material, such as an acrylic polymer (for example, PLEXIGLASS) or like material.

The facemasks 418, 420, as shown, include first ports 422, 424 configured to be operably coupled to a medical vacuum (with tubing 426, 428 and a filter 430, 432), second ports 434, 436 configured to be operably coupled to an air or oxygen supply (with a supply tube 438, 440 and a valve 442, 444), and third ports 446, 448 configured to be operated as an exhaust (with a filter 450, 452 and a valve 454, 456).

The facemasks 418, 420 include a protrusion (not shown) having a port (not shown) and cap 462, 463 (having two ports 464, 466, 468, 470 and associated filters 472, 474, 476, 478). The ports 464, 466, 468, 470 of the respective caps 462, 463 are configured to receive a medical instrument for conducting a medical procedure therethrough (one instrument 480, 482 is shown in each figure).

The particular embodiment of FIG. 17 also includes an intubation aperture 488. The intubation aperture 488 may be fabricated as walls 172 (FIG. 6) extending away from a surface 174 (FIG. 6) of the mask 122 (FIG. 6) and defining an opening therebetween, may be cut into the facemask 420 after fabrication and, therefore, not incorporate walls, or fabricated in another way that that would understood by those of ordinary skill in the art having the benefit of this disclosure. The intubation aperture 488 may be configured to receive a window 490 comprising a frame 492 and a sheath 494 stretched within the frame 492. The window 490 may be secured by friction fit and sealed with an O-ring 182 (FIG. 6) (or a c-ring, for example), include a rail and channel snap fit, or tabs received by recesses (not shown). The sheath 494 may be any material (such as a membrane) that is self-healing while permitting passage of an endotracheal tube.

While not specifically illustrated herein, it would be readily appreciated that another embodiment of a negative pressure hood (not shown) may incorporate instrument ports 226, 228 fabricated in a manner similar to the mask 192 of FIG. 11 and without the protrusion 90 and the cap 92 of FIG. 2. Moreover, it would be understood that certain elements and features of the various embodiment provided herein may be combined or removed as needed for providing protection to the health care worker during medical procedures.

The illustrated embodiments herein show valves at the surface of the masks; however, it would be understood by the skilled artisan that valve may be positioned along a tubing and proximate to the respective mask. For some of these embodiments, a distal end of the tubing may be molded for affixed to the surface of the respective mask while a proximal end of the tubing may include an adaptor configured to receive the valve.

Furthermore regarding valves, it would be understood that other valves may be incorporated into one or more embodiments and that the number and types of valves are not limited to those particularly disclosed herein. For example, ports associated with exhalation may include a positive end expiratory pressure valve that is configured to regulate a pressure within the patient's lung to improve lung inflation. This particular valve may have particular benefit in maintaining pressure on lower airways and an end of the breathing cycle to prevent alveoli collapse.

Additionally, it would be understood that the ports, windows, and other structures may be shaped or sized according to a particular surgical or fabrication need or requirement. For instance, the window 176 of FIGS. 6 and 7 is illustrated as being square in shape; however, those of ordinary skill in the art would appreciate that circular, oval, or rectangularly shaped windows could also be used.

Moreover, it would be understood that the sensor for airborne pathogens may be limited to a position within the filters of ports associated within air inflow or exhalation. Instead, the sensor may be incorporated into the mask, associated with the third port through which exhaled air is exhausted, and so forth.

Shapes and sizes of the various embodiments may vary from the illustrated examples provided here. For example, pediatric oronasal apparatuses may require one arrangement of ports while geriatric oronasal apparatuses require another.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept

What is claimed is:

1. A negative pressure surgical apparatus comprising:
a mask configured to cover a portion of the face of a patient;
a first port extending through the mask and configured to draw negative pressure from between the mask and the face of the patient; and
an instrument port extending through the mask and configured to receive a medical instrument therethrough and form a seal therewith, the instrument port positioned on the mask so as to permit surgery at a surgical site.

2. The negative pressure surgical apparatus of claim 1, further comprising:
a filter operably coupled to the first port and configured to detect airborne pathogens, removed airborne pathogens, or both.

3. The negative pressure surgical apparatus of claim 1, further comprising:
wherein the first port further includes a valve operably coupled thereto and configured to control a flow of gas to one direction.

4. The negative pressure surgical apparatus of claim 1, further comprising:
a second port extending through the mask and configured to provide a gas to the patient.

5. The negative pressure surgical apparatus of claim 4, wherein the second port further includes a filter operably coupled thereto and configured to detect airborne pathogens, remove airborne pathogens, or both.

6. The negative pressure surgical apparatus of claim 4, wherein the second port further includes a valve operably coupled thereto and configured to limit a flow of gas to one direction.

7. The negative pressure surgical apparatus of claim 1, further comprising:
a third port extending through the mask and configured to exhaust exhaled breath of the patient.

8. The negative pressure surgical apparatus of claim 7, wherein the third port further includes a filter operably coupled thereto and configured to detect airborne pathogens, remove airborne pathogens, or both.

9. The negative pressure surgical apparatus of claim 7, wherein the third port further includes a valve operably coupled thereto and configured to limit a flow of gas to one direction.

10. The negative pressure surgical apparatus of claim 1, further comprising:
a cuff operably coupled to a perimeter of the mask and configured to form the seal between the mask and the face of the patient.

11. The negative pressure surgical apparatus of claim 1, further comprising:
a sensor configured to detect a presence of an airborne pathogen.

12. The negative pressure surgical apparatus of claim 1, further comprising:
a cap having the instrument port therein; and
a protrusion extending from a surface of the mask, configured to removably receive the cap, and having an opening corresponding to the instrument port of the cap.

13. The negative pressure surgical apparatus of claim 1, further comprising:
an intubation aperture extending through the mask and configured to receive an endotracheal intubation tube and form a seal therewith.

14. The negative pressure surgical apparatus of claim 13, wherein the intubation aperture is configured to receive a sheath configured to form the seal with the endotracheal intubation tube, to seal the intubation aperture, or both.

15. The negative pressure surgical apparatus of claim 1, wherein the portion of the face of the patient covered by the mask includes the nose.

16. The negative pressure surgical apparatus of claim 15, wherein the portion further includes the mouth.

17. The negative pressure surgical apparatus of claim 1, further comprising:
a strap configured to secure the mask to the face of the patient.

18. A biohazard suit comprising:
a hood; and
the negative pressure surgical apparatus of claim 1 operably coupled to the hood.

19. A negative pressure surgical apparatus comprising:
a mask configured to cover a portion of the face of a patient;
a first port extending through the mask and configured to draw negative pressure from between the mask and the face of the patient;
a second port extending through the mask and configured to provide a gas to the patient;
a third port extending through the mask and configured to exhaust exhaled breath of the patient; and
an instrument port extending through the mask and configured to receive a medical instrument therethrough and form a seal therewith, the instrument port positioned on the mask so as to permit surgery at a surgical site.

20. The negative pressure surgical apparatus of claim 19, further comprising:
a filter configured to detect airborne pathogens, remove airborne pathogens, or both, the filter being operably coupled to the first port, the second port, the third port, or a combination thereof.

21. The negative pressure surgical apparatus of claim 19, further comprising:
a valve configured to limit a flow of gas to one direction, the valve being operably coupled to the first port, the second port, the third port, or a combination thereof.

22. The negative pressure surgical apparatus of claim 19, further comprising:
a cuff operably coupled to a perimeter of the mask and configured to form the seal between the mask and the face of the patient.

23. The negative pressure surgical apparatus of claim 19, further comprising:
a sensor configured to detect a presence of an airborne pathogen.

24. The negative pressure surgical apparatus of claim 19, further comprising:
a cap having the instrument port therein; and
a protrusion extending from a surface of the mask, configured to removably receive the cap, and having an opening corresponding to the instrument port of the cap.

25. The negative pressure surgical apparatus of claim 19, further comprising:
an intubation aperture extending through the mask and configured to receive an endotracheal intubation tube and form a seal therewith.

26. The negative pressure surgical apparatus of claim 25, wherein the intubation aperture is configured to receive a sheath configured to form the seal with the endotracheal intubation tube, to seal the intubation aperture, or both.

27. The negative pressure surgical apparatus of claim 19, wherein the portion of the face of the patient covered by the mask includes the nose.

28. The negative pressure surgical apparatus of claim 27, wherein the portion further includes the mouth.

29. The negative pressure surgical apparatus of claim 19, further comprising:
a strap configured to secure the mask to the face of the patient.

30. A biohazard suit comprising:
a hood; and
the negative pressure surgical apparatus of claim 19 operably coupled to the hood.

31. A method of performing oronasal surgery, the method comprising:
positioning a negative pressure surgical apparatus on the face of a patient, the negative pressure surgical apparatus comprising:
a mask configured to cover a portion of the face of the patient;
a first port extending through the mask; and
an instrument port extending through the mask and configured to receive a medical instrument therethrough and form a seal therewith, the instrument port positioned on the mask so as to permit surgery at a surgical site;
forming a seal between the face of the patient and the mask by drawing negative pressure through the first port; and
inserting the medical instrument through the instrument port to the surgical site.

32. The method of claim 31, further comprising:
filtering air drawn by the negative pressure through the first port.

33. The method of claim 31, where the negative pressure surgical apparatus includes a second port extending through the mask, the method further comprising:
providing a gas to the patient through the second port.

34. The method of claim 33, further comprising:
filtering the gas provided to the patient through the second port.

35. The method of claim 31, wherein the negative pressure surgical apparatus includes a third port extending through the mask, the method further comprising:
exhausting patient exhaled breath through the third port.

36. The method of claim 35, further comprising:
filtering the exhaled breath.

37. The method of claim 31, wherein the negative pressure surgical apparatus includes an intubation aperture extending through the mask, the method further comprising:
intubating the patient through the intubation aperture.
